(19) United States
(12) Patent Application Publication
(10) Pub. No.: US 2014/0186929 A1
(43) Pub. Date: Jul. 3, 2014

(54) COMPOSITIONS AND METHODS COMPRISING A COMBINATION SILAGE INOCULANT

(71) Applicants: E.I. DU PONT DE NEMOURS AND COMPANY, Wilmington, DE (US); PIONEER HI-BRED INTERNATIONAL, INC., Johnston, IA (US); DUPONT NUTRITION BIOSCIENCES APS, Copenhagen K (DK)

(72) Inventors: Ellen Davis, Johnston, IA (US); Keith Mertz, Johnston, IA (US); Terry Parrott, Johnston, IA (US); Barbara G. Ruser, Buxtehude (DE); William Rutherford, Grimes, IA (US); Brenda Smiley, Granger, IA (US); Annette Spielbauer, Johnston, IA (US); Ardean Veldkamp, Johnston, IA (US)

(21) Appl. No.: 13/827,207

(22) Filed: Mar. 14, 2013

Related U.S. Application Data
(60) Provisional application No. 61/746,912, filed on Dec. 28, 2012.

(51) Int. Cl. C12P 5/02

(52) U.S. Cl. CPC C12P 5/023
USPC 435/252.4; 435/167; 585/16

(57) ABSTRACT

Compositions and methods for the production of biogas from forage are provided. Compositions comprise a combination microbial inoculant, silage produced from forage inoculated with the combination microbial inoculant, and biogas produced from the silage. Various methods are provided for increasing biogas production and decreasing dry matter loss by inoculating forage with a combination inoculant. In certain embodiments, inoculating forage with specific combinations of bacterial strains results in a synergistic decrease in dry matter loss and a synergistic increase in biogas production. In other embodiments, inoculating a biomass composition comprising silage and sludge with specific combinations of bacterial strains results in a synergistic increase in biogas production.

### CROSS-REFERENCE TO RELATED APPLICATION

This application is a non-provisional application which claims the benefit of U.S. Provisional Application No. 61/746,912, filed Dec. 28, 2012, which is hereby incorporated by reference in its entirety.

### FIELD OF THE INVENTION

The present invention relates to the field of microbial inoculants for silage and biogas production.

### BACKGROUND OF THE INVENTION

Biogas is produced when bacteria anaerobically convert organic matter to methane and can be used as a low-cost and renewable fuel for a variety of uses including heating, electricity, or as a fuel for motor vehicles. It is considered to be a low-grade natural gas, as it contains approximately from 50-65% methane. For farm-based biogas production, the primary organic matter source is manure, although research has shown that gas production can be greatly increased by adding additional substrates. For example, the most common substrate is derived from energy crops such as corn silage. However, difficulties in the utilization of fiber from energy crops have been shown to be a limiting factor in the efficient production of biogas.

There are significant intrinsic similarities between the need for improved digestion of silage in the rumen of an animal and anaerobic biogas generation. The successful enhancement of fiber digestion in the rumen of an animal results in the animal obtaining increased nutrients from its feed. As a result, the animal demonstrates increased milk yield in dairy cows, and beef production in forage fed animals. Accordingly, farmers either tolerate a lower level of feed digestibility from silage, and therefore productivity, or use inoculants, forage additives or other feed additives to improve digestibility of feed. As a result of the similar approaches to degradation of plant fiber, the biogas industry has considered non-traditional methods for improving the production of biogas from energy crops such as corn silage.

Identification of combinations of microorganisms that can work together to break down the parts of plant cells that are difficult to digest could lead to significant increases in animal feed digestion and the production of biogas.

### BRIEF SUMMARY OF THE INVENTION

Compositions and methods for the production of biogas from forage are provided. Compositions comprise a combination microbial inoculant, silage produced from forage inoculated with the combination microbial inoculant, and biogas produced from the silage. Various methods are provided for increasing biogas production and decreasing dry matter loss by inoculating forage with a combination inoculant. In other embodiments, inoculating a biomass composition comprising silage and sludge with specific combinations of bacterial strains results in a synergistic increase in biogas production.

### BRIEF DESCRIPTION OF THE DRAWINGS

FIG. 1 demonstrates the production of biogas from whole plant corn silage treated with a combination microbial inoculant, treated with individual bacterial cultures X11M58 and 11CH4, or from untreated whole plant corn silage.

FIG. 2 reports the production of methane from whole plant corn silage treated with a combination microbial inoculant, treated with individual bacterial cultures X11M58 and 11CH4, or from untreated whole plant corn silage.

### DETAILED DESCRIPTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### I. Overview

As used herein, the term “biogas” refers to a gaseous material produced by the anaerobic digestion or fermentation of organic matter. Typically, biogas is a mixture of primarily methane and carbon dioxide gases. In some embodiments disclosed herein, biogas comprises at least about 40%, at least about 50%, at least about 60%, at least about 65%, at least about 70%, at least about 75%, at least about 80%, at least about 85%, at least about 90%, at least about 95%, at least about 100%, at least about 30-90%, at least about 50-90%, or at least about 60%-80% methane gas.

“Biomass” or “biomass composition”, as used herein, refers to the organic matter used as a substrate for anaerobic digestion or fermentation to produce biogas. Biomass can contain sludge, silage, a combination of both sludge and silage, or any other digestible organic matter. In some embodiments, biomass comprises a combination of sludge and silage, wherein silage accounts for at least about 1%, at least about 2%, at least about 3%, at least about 4%, at least about 5%, at least about 10%, at least about 15%, at least about 20%, at least about 25%, at least about 30%, at least about 40%, at least about 50%, at least about 60%, at least about 70%, at least about 80%, or at least about 90% of the total biomass composition. Biomass compositions disclosed herein can also comprise a microbial inoculant or combination microbial inoculant as disclosed elsewhere herein.

As used herein, the term “sludge”, or “slurry”, comprises manure from a single type of animal or a mixture of manure from different types of animals. For example, sludge or seeding sludge can contain cow manure, swine manure, poultry (i.e., chicken, turkey, or duck) manure, horse manure, rabbit manure, or any combination thereof. In certain embodiments, sludge contains at least about 0%, at least about 25%, at least about 40%, at least about 50%, at least about 60%, at least about 65%, at least about 70%, at least about 75%, at least about 80%, at least about 90%, or at least about 100% cattle manure. In some embodiments, sludge contains at least about 0%, at least about 10%, at least about 20%, at least about 25%, at least about 30%, at least about 35%, at least about 40%, at least about 50%, at least about 60%, at least about 75%, or at least about 100% swine manure. Sludge can comprise sludge remaining after previous anaerobic digestion, such as that obtained from an anaerobic digester during or after biogas production, herein referred to as seeding sludge. In certain embodiments, microorganisms are added to sludge to assist in the digestion of organic matter. Microorganisms added to sludge can comprise any microorganism or bacterial culture described elsewhere herein. In some embodiments, a combination microbial inoculant is added to sludge prior to biogas production.

As set forth elsewhere herein, biomass may contain both sludge and silage. “Silage”, as used herein, refers to any type of fermented plant or plant part including, but not limited to, fermented grass, clover, alfalfa, wheat, legumes, beans, sunflower, barley, oats, triticale, soybean, whole plant corn silage (WPCS), sorghum, radish, artichoke, peas, sugar beets, fermented grains and grass mixtures, or any combination thereof. “Pre-ensiled plant material” or “forage” refers to any plant or plant part prior to undergoing the ensiling process including, but not limited to grass, clover, alfalfa, wheat, legumes, beans, sunflower, barley, oats, triticale, soybean, whole plant corn, corn stover, sorghum, radish, artichoke, peas, sugar beets, or any combination thereof. The plant and/or plant part may be freshly harvested or previously harvested and wilted or partially dried. Forage may be pre-processed by mechanical or other means including, but not limited, to chopping, cutting, milling, slicing or any suitable method of portioning the biomass and reducing particle size for silage production. For example, forage can be chopped to a theoretical chop length of about 10-13 mm.

### II. Microbial Inoculant

Compositions disclosed herein include microbial inoculants for use in the production of silage, biogas, and animal feed. Microbial inoculants as described herein can include a combination of bacterial strains containing ferulate esterase and proteolytic and fibrolytic enzymes. The fermentation of silage with microbial inoculants containing ferulate esterase producing organisms has been shown to enhance the digestibility of fiber in ruminants and enhance the production of biogas under anaerobic conditions. See, for example, U.S. Pat. No. 7,799,551 and U.S. Pat. No. 7,919,683, herein incorporated by reference in their entirety. Further, the addition of microorganisms that contain proteolytic and/or fibrolytic enzymatic activity can improve manure degradation, reduce odor, and enhance the production of biogas.

As used herein, “microbial inoculant” refers to a composition comprising at least one bacterial culture and a suitable carrier. A “combination microbial inoculant” comprises at least 2, at least 3, at least 4, at least 5, at least 6, at least 7, or more bacterial cultures and a suitable carrier. Bacterial cultures comprise at least one bacterial strain and may comprise multiple bacterial strains, including for example, at least 2, at least 3, at least 4, at least 5, at least 6, at least 7, or more. Bacterial cultures useful in the methods and compositions disclosed herein include, but are not limited to, X11M58 and 11CH4.

Bacterial strains for use in the microbial inoculant can contain ferulate esterase and/or proteolytic and fibrolytic enzymes. In some embodiments, bacterial strains for use in the microbial inoculant include but are not limited to: Lactobacillus buchneri, Bacillus licheniformis, and Bacillus subtilis, and derivatives thereof. Derivatives of bacterial strains disclosed herein comprise genetic alterations such as additions or deletions of polynucleotides such that the ability of a bacterial strain to produce biogas, increase biogas production, reduce dry matter loss, or enhance digestion of silage is not altered. Activity can be determined by any appropriate method described elsewhere herein.

In certain embodiments, a combination microbial inoculant comprises a first bacterial culture comprising a Lactobacillus, for example, L. buchneri deposited as PTA-6138, or derivatives thereof, and a second bacterial culture comprising a Bacillus culture. In specific embodiments, the second bacterial culture comprises a combination of Bacillus licheniformis BL842 deposited as NRRL B-50516, or derivatives thereof, Bacillus licheniformis BL21 deposited as NRRL B-50134, or derivatives thereof, and Bacillus subtilis BS27 deposited as NRRL B-50105, or derivatives thereof. See, for example, U.S. Pat. Nos. 7,754,469, 8, 021,654, and 8,025,874, herein incorporated by reference in their entirety. L. buchneri deposited as PTA-6138 can also be referred to as “Lactobacillus buchneri”, “strain LN4017”, “11CH4”, or “Pioneer 11CH4”. See, for example, U.S. Pat. No. 7,799,551, herein incorporated by reference in its entirety. As used herein “X11M58”, “11M58”, “M58”, “Microsource”, or “Accelerator D” refers to a Bacillus culture comprising about 70% B. licheniformis deposited as NRRL B-50516, about 20% B. licheniformis deposited as NRRL B-50134, and about 10% B. subtilis deposited as NRRL B-50105. In certain embodiments, the combination microbial inoculant comprises about equal parts 11CH4 and X11M58 or the microbial inoculant can comprise 11CH4 and X11M58 in a ratio of about 0.5:1, about 0.75:1, about 1:1, about 1.25:1, about 1.5:1, or about 2:1.

Suitable carriers for use in the microbial inoculants disclosed herein can be liquid or solid carriers. For example, solid carriers may be made up of calcium carbonate, starch, cellulose and combinations thereof. In one embodiment, carriers include Baker's sugar, maltodextrin M100, and baylith. Liquid carriers include, but are not limited to, water. The bacterial cultures and strains may be in any form suitable for addition to forage or a biomass composition. For example, bacterial cultures and strains may be in the form of a fresh live culture, rehydrated lyophilized bacterial cells or spores, or thawed frozen bacterial preparation.

### III. Methods of Biogas Production

Methods for the production of biogas from silage are provided comprising adding a microbial inoculant to forage, ensiling the forage inoculated with the microbial inoculant to produce silage, and adding biomass comprising the silage to a biogas generator, wherein biogas is produced in the biogas generator. An effective amount of any microbial inoculant, or combination thereof, can be added to forage in the generation of silage for use in biogas production. As used herein, addition of an effective amount of a microbial inoculant to forage results in an increase in the production of biogas when compared to biogas produced from uninoculated forage. As used herein, “uninoculated forage” or “uninoculated silage” refers to forage or silage, respectively, that has been produced without the addition of a microbial inoculant or a combination microbial inoculant. In some embodiments addition of an effective amount of microbial inoculant to forage results in a synergistic increase in the production of biogas, or synergistic production of biogas.

Alternatively, methods for the production of biogas from a biomass composition are provided comprising inoculating a biomass composition comprising silage and sludge with an effective amount of a microbial inoculant and adding the inoculated biomass composition to a biogas generator, wherein biogas is produced in the biogas generator. An effective amount of a microbial inoculant added to a biomass composition results in an increase in the production of biogas when compared to biogas produced from an uninoculated biomass composition. In some embodiments addition of an effective amount of microbial inoculant to a biomass composition results in a synergistic increase in the production of biogas, or synergistic production of biogas.

“Synergy”, “synergistic”, “synergistically”, and derivations thereof, as used herein refer to circumstances under which biogas production from a substrate inoculated with a combination microbial inoculant is greater than the sum of biogas production from a substrate inoculated with the individual bacterial cultures used in the combination microbial inoculant. Synergistic biogas production can occur when forage is used as the inoculated substrate, and synergistic biogas production can occur when a biomass composition is used as the inoculated substrate. The combination microbial inoculant can comprise two or more different bacterial cultures as disclosed elsewhere herein.

Biogas production can refer to the total amount of biogas produced or the rate of biogas production. Biogas production can be measured 1 day, 5 days, 6 days, 7 days, 8 days, 9 days, 10 days, 11 days, 12 days, 13 days, 14 days, 15 days, 16 days, 17 days, 18 days, 19 days, 20 days, 25 days, 30 days, 40 days, 50 days, 60 days, or any other acceptable time after addition of biomass to the biogas generator. The rate of biogas production can be calculated over any time period before, during, or following fermentation of the biomass. For example, the rate of biogas production can be calculated from 0-5 days, 0-6 days, 0-8 days, 0-10 days, 0-12 days, 0-15 days, 5-6 days, 5-8 days, 5-10 days, 5-12 days, 5-15 days, 10-15 days, or any other time period following addition of biomass to the biogas generator.

A synergistic increase in biogas production can refer to an increase in production of biogas from forage inoculated with a first bacterial culture and a second bacterial culture that is greater than the sum of biogas production from forage inoculated with a first bacterial culture and a second bacterial culture individually, when compared to biogas production from uninoculated forage. In some embodiments, a synergistic increase in biogas production can refer to an increase in the rate of biogas production from forage inoculated with a first bacterial culture and a second bacterial culture that is greater than the sum of the rates of biogas production from forage inoculated with a first bacterial culture and a second bacterial culture individually, when compared to the rate of biogas production from uninoculated forage from 0-9 days following addition of biomass to the biogas generator.

The microbial inoculant as described elsewhere herein can be added to forage by any method suitable for proper mixture of the inoculant with the plant material. For example, the inoculant can be sprayed onto the forage prior to ensiling or as the material is being ensiled.

As used herein, “ensiling” or “ensiled” refers to an anaerobic fermentation process used to preserve forages, immature grain crops, and other biomass crops for feed and biofuels. In some embodiments, the process of ensiling comprises the steps of contacting forage with a microbial inoculant and storing the mixture in an anaerobic condition. In certain embodiments, the process of ensiling comprises the steps of storing forage in anaerobic condition in a manner so as to exclude air. Forage, having been inoculated with the microbial inoculant described elsewhere herein, is also packed and stored in a manner so as to exclude air. The moisture content of forage can be about 50% to about 80%, depending on the means of storage, the amount of compression, and the expected moisture loss during storage. Ensiling can occur in silos, silage heaps, silage pits, silage bales, or any other method appropriate for ensiling the chosen plant material. Plant material with the microbial inoculant described elsewhere herein can be ensiled for any amount of time appropriate to produce silage at the desired maturity stage. In some embodiments, ensiling occurs for about 15, about 20, about 25, about 30, about 35, about 40, about 41, about 42, about 43, about 44, about 45, about 46, about 47, about 48, about 49, about 50, about 55, about 60, about 65, about 70 days, about 4 months, about 8 months, about 12 months, about 18 months, or about 24 months. The ensiling process can take place at any ambient temperature, for example at an ambient temperature from 0-45° C. The temperature of the plant material being ensiled may, however, increase above 45° C. Mature silage can be used for animal feed, frozen and stored for a later use, or added to a biogas generator for the production of biogas.

In certain embodiments of the invention, the microbial inoculant is added to forage at a concentration of about 10¹, about 10², about 10³, about 10⁴, about 10⁵, about 10⁶, about 10⁷, about 10⁸, about 10⁹, about 10 ¹⁰, about 10¹¹, or about 10¹² viable organisms per gram of forage. In specific embodiments, the microbial inoculant disclosed herein comprises about 10¹ to about 10¹², 10¹ to about 10¹¹, 10¹ to about 10¹⁰, about 10² to about 10⁹, about 10² to about 10⁸, about 10³ to about 10⁶, or about 10⁴ to about 10⁵ viable organisms of 11CH4 per gram of forage, combined with about 10¹ to about 10¹², 10¹ to about 10¹¹, 10¹ to about 10¹⁰, about 10² to about 10⁹, about 10² to about 10⁸, about 10³ to about 10⁶, or about 10⁴ to about 10⁵ viable organisms of X11M58 per gram of forage. Where more than one strain is used in the microbial inoculant, the concentration can be calculated for each strain, for each bacterial culture, or for the combination microbial inoculant.

The amount of plant material that is lost due to aerobic degradation resulting from oxygen trapped at the beginning of the ensiling process or due to oxygen introduced during silo unloading is referred to as “aerobic dry matter loss”, “aero DML”, “dry matter loss”, or simply “DML”. Total dry matter loss during the ensiling process includes aerobic dry matter loss and dry matter lost due to inefficient fermentation of plant material. In some embodiments, inoculation of forage with an effective amount of a combination microbial inoculant results in a synergistic decrease in dry matter loss. Addition of an effective amount of a combination microbial inoculant results in a synergistic decrease in dry matter loss. As used herein, a synergistic decrease in dry matter loss occurs when the decrease in dry matter loss observed from ensiling forage inoculated with a combination microbial inoculant is greater than the additive decrease in dry matter loss from ensiling forage inoculated with the individual bacterial cultures used in the combination microbial inoculant. Dry matter loss can be calculated at the end of the ensiling process or any time during the ensiling process. Methods for calculating dry matter loss are known and include the methods described in Honig, H., (1985) Das Wirtschaftseigene Futter 21: 25-32, herein incorporated by reference in its entirety.

Following ensiling, biomass comprising silage is added to a biogas generator in order to produce biogas. As described elsewhere herein, biomass added to the biogas generator can comprise silage and seeding sludge. In some embodiments, silage produced from uninoculated forage is combined with sludge and a combination microbial inoculant prior to biogas production. In other embodiments, silage produced from uninoculated forage is combined with sludge having been inoculated with a combination microbial inoculant prior to biogas production. Alternatively, a combination microbial inoculant can be added directly to silage produced from uninoculated forage, prior to combination with sludge and subsequent production of biogas. In other embodiments, biomass comprising silage produced from forage inoculated with a microbial inoculant described herein is combined with sludge and added to a biogas generator in order to produce biogas.

In certain embodiments of the invention, the microbial inoculant is added to a biomass composition at a concentration of about 10¹, about 10², about 10³, about 10⁴, about 10⁵, about 10⁶, about 10⁷, about 10⁸, about 10⁹, about 10¹⁰, about 10¹¹, or about 10¹² viable organisms per gram of biomass composition. In specific embodiments, the microbial inoculant disclosed herein comprises about 10¹ to about 10¹², 10¹ to about 10¹¹, 10¹ to about 10¹⁰, about 10² to about 10⁹, about 10² to about 10⁸, about 10³ to about 10⁶, or about 10⁴ to about 10⁷ viable organisms of 11CH4 per gram of biomass composition, combined with about 10¹ to about 10¹², 10¹ to about 10¹¹, 10¹ to about 10¹⁰, about 10² to about 10⁹, about 10² to about 10⁸, about 10³ to about 10⁶, or about 10⁴ to about 10⁷ viable organisms of X11M58 per gram of biomass composition. Where more than one strain is used in the microbial inoculant, the concentration can be calculated for each strain, for each bacterial culture, or for the combination microbial inoculant.

A synergistic increase in biogas production can refer to an increase in production of biogas from a biomass composition inoculated with a first bacterial culture and a second bacterial culture that is greater than the sum of biogas production from a biomass composition inoculated with a first bacterial culture and a second bacterial culture individually, when compared to biogas production from a biomass composition not having the first bacterial culture or the second bacterial culture. In some embodiments, the biomass composition used in the methods disclosed herein comprises silage produced from uninoculated forage.

The biogas generator, or anaerobic digester, can be constructed and used according to standard methods. Anerobic digestion of the biomass can be in a batch process or continuous process. In continuous biogas production, the anaerobic digester can be fed each day with new biomass. New biomass can be added from 1-12 times/day. In some embodiments, new biomass can be added every 2 hours at a rate of 2% (volume) new biomass/day. The new biomass may be the same as or different from the biomass that is used to initiate the biogas production process. For example, a mixture of sludge and silage produced from forage inoculated with a microbial inoculant can be used as the initial biomass at the beginning of a biogas production process. Once the process has been started and is running, silage and/or fresh plant or plant part may be added to continue anaerobic digestion and biogas formation. Fermentation in the biogas generator can be carried out at any temperature that produces biogas. In specific embodiments, fermentation is carried out at a mesophilic temperature of 38-40° C.

Volume of biogas produced can be measured directly, or calculated from a mathematical model. Any mathematical model can be used to calculate biogas volume, including, but not limited to, Simple Exponential Growth with Cut-off, Substrate Limited Exponential Growth, Logistic Growth, and Gompertz Growth. Any chosen mathematical model can have either a single-pool approach or a dual-pool approach. See, for example, Schofield, P., et al. (1994) J. Anim. Sci. 72: 2980-2991, herein incorporated by reference in its entirety. After collection, biogas can be used for any purpose such as direct addition to a gas grid or used to generate electricity.

### IV. Biological Deposits

The strains indicated below were deposited with the Agricultural Research Service (ARS) Culture Collection, housed in the Microbial Genomics and Bioprocessing Research Unit of the National Center for Agricultural Utilization Research (NCAUR), under the Budapest Treaty provisions. The strains were given the indicated accession numbers. The address of NCAUR is 1815 N. University Street, Peoria, Ill., 61604. The deposits will irrevocably and without restriction or condition be available to the public upon issuance of a patent. However, it should be understood that the availability of a deposit does not constitute a license to practice the subject invention in derogation of patent rights granted by government action:

Bacillus licheniformis BL842 deposited as NRRL B-50516 on Apr. 15, 2008

Bacillus licheniformis BL21 deposited as NRRL B-50134 on May 20, 2011, and

Bacillus subtilis BS27 deposited as NRRL B-50105 on Jan. 24, 2008.

A deposit of the following microbial strain has been made with the American Type Culture Collection (ATCC), 10801 University Blvd., Manassas, Va. 20110-2209:

Lactobacillus buchneri LN4017 (ATCC Accession No. PTA-6138. This strain was deposited on Aug. 3, 2004. The strain deposited with the ATCC was taken from the same deposit maintained at Pioneer Hi-Bred International, Inc. (Des Moines, Iowa). Applicant(s) will meet all the requirements of 37 C.F.R. §§1.801-1.809, including providing an indication of the viability of the sample when the deposit is made. Each deposit will be maintained without restriction in the ATCC Depository, which is a public depository, for a period of 30 years, or 5 years after the most recent request, or for the enforceable life of the patent, whichever is longer, and will be replaced if it ever becomes nonviable during that period. The deposits will irrevocably and without restriction or condition be available to the public upon issuance of a patent. However, it should be understood that the availability of a deposit does not constitute a license to practice the subject invention in derogation of patent rights granted by government action.

The article “a” and “an” are used herein to refer to one or more than one (i.e., to at least one) of the grammatical object of the article. By way of example, “an element” means one or more elements.

All publications and patent applications mentioned in the specification are indicative of the level of those skilled in the art to which this disclosure pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

### Experimental

### EXAMPLE 1

### Silage Production Using Combination Microbial Inoculant

Microbial inoculants used in this study had the following composition:
11CH4—Lactobacillus buchneri deposited as PTA-6138 (100%)
X11M58—Bacillus licheniformis deposited as NRRL B-50516 (70%)
   Bacillus licheniformis deposited as NRRL B-50134 (20%)
   Bacillus subtilis deposited as NRRL B-50105 (10%).

Whole plant corn forage (approximately 30% dry matter (DM)) was harvested for three trials using a precision forage chopper to a theoretical chop length of about 10-13 mm.

The individual inoculants were applied to supply at a rate of approximately 1×10⁵ cfu per gram forage for 11CH4 and 1×10⁵ cfu per gram forage for X11M58. The combination inoculant was applied at a rate equal to the individual treatments (1×10⁵ cfu/g forage 11CH4 and 1×10⁵ cfu/g forage X11M58). All treatments were applied by syringe and thoroughly mixed into the forage by rolling on clean plastic sheeting. For each treatment, four experimental 10×36 cm PVC silos were filled with sufficient forage to give a density of approximately 150 kg DM/cubic meter. Silos were stored until opening in a climate controlled chamber at 20 degrees Celsius.

Upon opening, sub-samples of forage were taken for DM determination and aqueous extracts were prepared for HPLC analysis of volatile fatty acids. DM losses were calculated and aerobic stability was determined by the methods described by Honig, H., (1985) Das Wirtschaftseigene Futter 21: 25-32. The remaining forage was frozen for use as a substrate for biogas production.

The treatment of whole plant corn forage with strains of 11CH4 or X11M58 alone as well as the combination of the two, produced quality silage after 60 days of fermentation. Table 1 compares the effect of these treatments on quality of whole plant corn silage with an uninoculated control. The high content of lactic acid results in good preservation of forage in all treatments as evidenced by low fermentation DM losses.

Aerobic stability of the silage treated with all individual products or a combination was good. Compared to control, both X11M58 and the combination of 11CH4 and X11M58 provided nearly a 24 hour increase in aerobic stability over the uninoculated control. In addition to the increased length of time prior to heating, the effects of microbial activity are minimized as shown by the nearly 2% unit decrease in aerobic DM losses with the combination of the two products.

TABLE 1
Effect of Inoculant Treatments on Silage Quality in Whole Plant
Corn Forage (summary of three trials)

| | | | | 11CH4 + |
|---|---|---|---|---|
| | Control | 11CH4 | X11M58 | X11M58 |
| DM % | 30.07 | 30.33 | 30.47 | 30.63 |
| pH | 3.87 | 3.83 | 3.81 | 3.80 |
| Lactic (% FM) | 1.86 | 1.86 | 1.93 | 1.88 |
| Acetic (% FM) | 0.80 | 0.80 | 0.83 | 0.80 |
| Ethanol (% FM) | 0.21 | 0.28 | 0.26 | 0.31 |
| Fermentation DM | 1.17 | 1.17 | 1.13 | 1.23 |
| loss (%) | | | | |
| Aerobic Stability | 109 | 107 | 121 | 128 |
| (h) | | | | |
| Aerobic DM Loss | 5.80 | 5.30 | 3.47 | 2.33 |
| (%) | | | | |
| Total DM Loss, % | 6.97 | 6.47 | 4.60 | 3.56 |
| (aerobic + anaerobic) | | | | |

The total dry matter loss during anaerobic and aerobic fermentation was decreased slightly by treatment with 11CH4 (˜0.5% units) and 2.3% units by treatment with X11M58. When silage was treated with both 11CH4 and X11M58 the total dry matter loss was improved to 3.5% units, nearly one unit higher than the sum of the effects of either product alone. For a typical ensiling system for biogas (˜10,000 tons silage produced per year) the 1% unit improvement with the products in combination translates into an addition 100 tons of silage to be utilized to fuel the biogas generator or an additional 5 days feed available.

### EXAMPLE 2

### Synergistic Production of Biogas Using Combination Microbial Inoculant

Estimates of biogas production were obtained from frozen silage according to the procedure described herein. Neutral density polyethylene containers of approximately 20 Liters were filled with 15 Liters of seeding sludge composed of 70% cattle manure and 30% swine manure. Each container was equipped with a valve in the lid to allow gas collection. To the 15 liter seeding sludge, 500 grams frozen silage material was added and the container was tightly sealed. Periodic mixing was accomplished with a mechanical mixer or by shaking of the container.

Resulting gas volumes produced were measured with a drum-type gas volume meter or by volumetric measurement prior to or after collection into a gas-tight analysis bag. Gas composition was measured with a Dräger X-am 7000 gas analyzer equipped to monitor carbon dioxide, methane and hydrogen sulfide. Composition readings were taken daily and the volume of methane generated was determined from the methane percentage of the collected gas.

Because discrete collection of data points is only casually related to biogas production and the production of biogas and methane is a direct result of microbial activity from different populations of organisms, mathematical models are used to interpret the data obtained. Given that gas production is a function of microbial population growth, it is feasible to utilize bacterial growth models to describe the continuous production of gasses. It is imperative that the model used to describe gas production has biological relevance and fits experimental data with a statistical degree of certainty. A single pool logistic model fits these criteria.

Experimental data was fit to a single pool logistic model having the generalized integrated form of:

V=V_{f}*[1+exp(2-4S(t−λ))]⁻¹

Where: ^{V}_{f}=maximum gas volume produced
λ=lag
S=specific fractional rate (maximum rate/maximum volume)

Data was fit to the single pool logistic model above utilizing TableCurve 2D (Jandel Scientific, San Rafael, Calif.) insuring that all curve fittings were not significantly different than the experimental data.

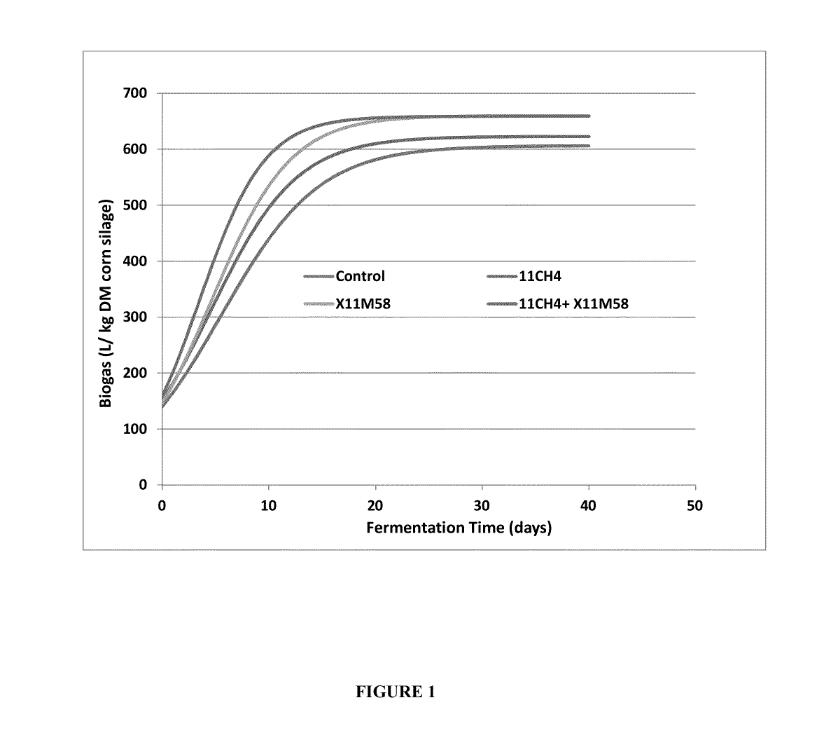

Treatment of corn forage with 11CH4, X11M58 or a combination of the two increased the production of biogas between 3 and 9% with X11M58 and the combination showing the greatest improvement in total biogas produced (FIG. 1). It can also be seen in FIG. 1 that the rate of biogas production is significantly enhanced by the addition of 11CH4 (14.7%) and X11M58 (24.4%). When whole plant corn forage is treated with a combination of 11CH4 and X11M58 the rate of biogas production is enhanced by nearly 10% over the sum of the individual treatments (Table 2).

TABLE 2
Effect of Inoculant Treatments on Biogas Production
(summary of three trials)

| | | | | 11CH4 + |
|---|---|---|---|---|
| | Control | 11CH4 | 11M58 | 11M58 |
| Maximum biogas | 606.4 | 622.8 | 660.1 | 658.9 |
| volume (L/kg DM) | | | | |
| Improvement over | — | 2.7% | 8.8% | 8.6% |
| uninoculated control | | | | |
| Specific fractional | 5.43 | 6.23 | 6.76 | 8.18 |
| rate (%) | | | | |
| Improvement over | — | 14.7% | 24.4% | 50.6% |
| uninoculated control | | | | |

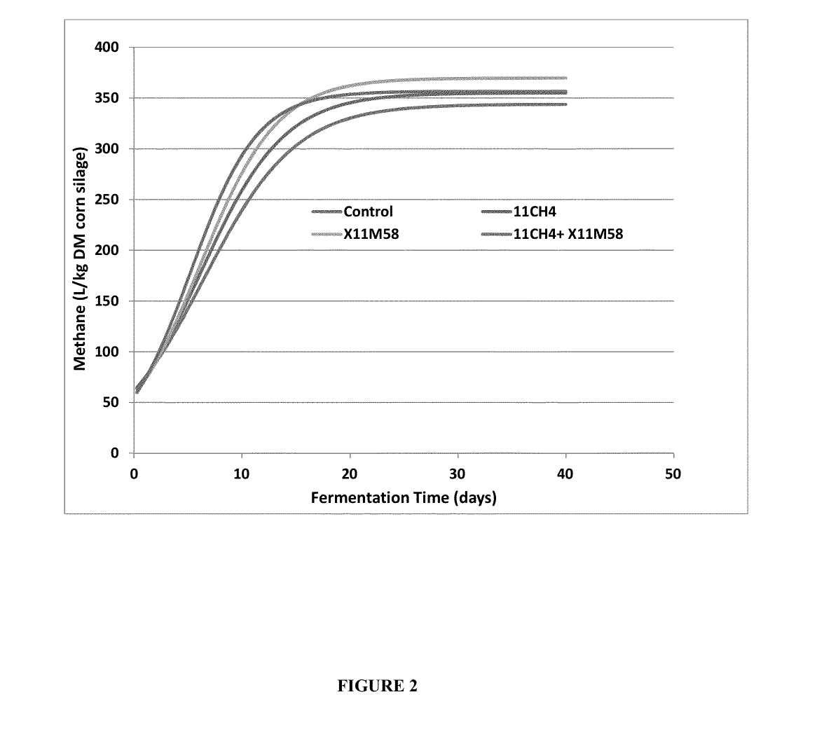

Similar results were obtained when methane was measured as a component of the biogas produced. (FIG. 2) All treatments improved the production of methane over the uninoculated control from 3.7-7.5%. The rate of biogas production was increased by all treatments of the whole plant corn forage. The treatment 11CH4 improved the rate by 9.66% while the X11M58 treatment increased the rate by 18.4%. (Table 3) When the two inoculants were added in combination the rate of methane production was 36.9%; a synergistic response of 8.8% more than the sum of the individual inoculants alone. The economic significance of such enhancements in methane production by treatment with silage treatments must consider both the amount of methane produced from each kilogram of silage and how quickly the methane is produced. Capital expenditures for biogas installations are very high. As with any multi-stage process, the returns depend not only on the amount of end product but how quickly the end product is produced. The use of 11CH4 and X11M58 together results in a considerable increase in rate over uninoculated control or either individual product used alone. The increased rate of methane production results in greater production per unit time allowing for maximum operation of the biogas system. Additionally, the improved dry matter recovery of the silage when the combination of the two products is used on a corn forage, results in additional feed available for conversion to methane.

TABLE 3
Effect of Inoculant Treatments on Methane Production
(summary of three trials)

| | | | | 11CH4 + |
|---|---|---|---|---|
| | Control | 11CH4 | 11M58 | 11M58 |
| Maximum methane | 343.8 | 355.0 | 369.6 | 356.6 |
| volume (L/kg DM) | | | | |
| Improvement over | — | 3.2% | 7.5% | 3.7% |
| uninoculated control | | | | |
| Specific fractional | 5.88 | 6.45 | 6.96 | 8.05 |
| rate (%) | | | | |
| Improvement over | — | 9.7% | 18.4% | 36.9% |
| uninoculated control | | | | |

### EXAMPLE 3

### Biogas Production Using Microbial Inoculants on the Manure Slurry

Microbial silage inoculant used in this study had the following composition:
11CH4—Lactobacillus buchneri, PTA-6138 (100%)
   Microbial slurry inoculant used in this study had the following composition:
X11M58—Bacillus licheniformis, NRRL B-50516 (70%)
   Bacillus licheniformis, NRRL B-50134 (20%)
   Bacillus subtilis NRRL B-50105 (10%)
11CH4—Lactobacillus buchneri, PTA-6138 (100%)

Silage Preparation

Whole plant corn forage (approximately 35% dry matter (DM)) was harvested using a precision forage chopper to a theoretical chop length of 10-13 mm. The 11CH4 inoculant was applied at a rate of approximately 10⁵ cfu per gram forage (wet weight) and thoroughly mixed into the forage; uninoculated control silage was also included. For each treatment, eight experimental packets (heat-seal bags) were filled with sufficient forage to give a density of approximately 150 kg DM/cubic meter using a professional grade food sealer. Silage packets were stored until opening in a climate controlled chamber at 20° C. Upon opening, sub-samples of forage were dried at 62° C. with forced air for 48 hrs and ground to 6 mm in a Wiley mill. The dried ground forages from all 6 studies were composited by treatment and used as a substrate for biogas production.

Biogas Testing Procedure

A 150 L manure seed slurry tank was anaerobically maintained at 30-35° C., supplied substrate and manure, and stirred periodically. Batch anaerobic fermentations were prepared by adding substrate or additives to 60 g of manure slurry (from seed slurry tank) to 250 ml serum vials sealed with rubber stoppers. Control or 11CH4-treated silages were supplied as substrate at a ratio of 30:1 manure slurry:substrate (wet weight), or approximately 0.6 g dried ground silage per vial. Microbial additives were applied to batch fermentation vials at combinations and doses listed in Table 4. Biogas production was measured with water displacement and expressed on a liter biogas/kg substrate basis. Gas measurements were taken periodically during the fermentation up to 15 days. Gas produced from control samples containing manure slurry without substrate was subtracted from test samples. Values were adjusted for slurry weight, substrate weight, and an assay standard (control silage with no microbials added to the slurry). Experimental data was fit to a single pool logistic model as described in the previous example.

TABLE 4
Microbial additives applied to manure slurry vials.
Dose values are cfu/g substrate (DM).

| | X11M58 | 11CH4 |
|---|---|---|
| None | 0 | 0 |
| Low | 2.17 × 10⁵ | 1.0 × 10⁴ |
| Medium | 2.17 × 10⁶ | 1.0 × 10⁵ |
| High | — | 1.0 × 10⁶ |

Results

The biogas fermentations with control silage as biomass substrate and supplemented with low/medium levels of X11M58 in combination with high levels of 11CH4 resulted in approximately 4.6% more biogas than the other fermentations given control silage (Table 5). In general, there is an increase in the amount of gas produced as one moves toward the highest doses. Specifically, a synergistic increase in the production of biogas is observed after addition of the combination inoculant. Addition of 11CH4 alone increased biogas production by 0.6% (high dosage) and addition of 11M58 alone increased biogas production by 0.4% (medium dosage). However, when 11CH4 (high dosage) and 11M58 (medium dosage) are added in combination, biogas production increases by 4.4%; a synergistic response of 3.4% more than the sum of the increases in biogas formation by the individual inoculants alone.

TABLE 5
Total biogas production from untreated silage supplemented with
X11M58 & 11CH4 at the slurry (sludge)

Volume of Biogas (l/kg substrate)

| | 11CH4 | 11CH4 | 11CH4 | 11CH4 |
|---|---|---|---|---|
| | None | Low | Medium | High |
| 11M58 None | 490 | 498 | 494 | 493 |
| 11M58 Low | 488 | 480 | 486 | 513 |
| 11M58 Medium | 492 | 491 | 490 | 512 |

In contrast, as the combination dose is increased, the rate of biogas formation is reduced (Table 6). This combination and dose of these particular bacterial strains produced conditions that were conducive to increased gas production, possibly by decreasing the rate of digestion thus allowing a more complete conversion of the added biomass to gas before conditions in the closed system become unfavorable for digestion.

TABLE 6
Biogas production rate from untreated silage supplemented with
X11M58 & 11CH4 at the slurry (sludge)

Specific Fractional Rate (%/hr)

| | 11CH4 | 11CH4 | 11CH4 | 11CH4 |
|---|---|---|---|---|
| | None | Low | Medium | High |
| 11M58 None | 9.700 | 8.770 | 9.270 | 9.230 |
| 11M58 Low | 9.660 | 8.960 | 8.440 | 8.470 |
| 11M58 Medium | 9.470 | 8.980 | 9.120 | 8.710 |

When 11CH4-treated silage was used as biomass substrate in the fermentations, the addition of inoculants to the slurry did not result in higher levels of gas production. In this case, increasing levels of inoculants generally decreased gas production (Table 7). Rates of biogas production were unaffected by the addition of the microbial treatments, either alone or in combination (Table 8).

TABLE 7
Total biogas production from 11CH4-treated silage
supplemented with X11M58 & 11CH4 at the slurry (sludge)

Volume of Biogas (l/kg substrate)

| | 11CH4 | 11CH4 | 11CH4 | 11CH4 |
|---|---|---|---|---|
| | None | Low | Medium | High |
| 11M58 None | 522 | 496 | 489 | 522 |
| 11M58 Low | 506 | 481 | 500 | 454 |
| 11M58 Medium | 488 | 497 | 455 | 467 |

TABLE 8
Biogas production rate from 11CH4-treated silage
supplemented with X11M58 & 11CH4 at the slurry (sludge)

Specific Fractional Rate (%/hr)

| | 11CH4 | 11CH4 | 11CH4 | 11CH4 |
|---|---|---|---|---|
| | None | Low | Medium | High |
| 11M58 None | 10.340 | 10.410 | 10.300 | 10.340 |
| 11M58 Low | 9.960 | 10.320 | 10.300 | 10.200 |
| 11M58 Medium | 10.390 | 10.080 | 10.560 | 10.460 |

As shown in the example, treatment of forage with 11CH4 prior to ensiling resulted in a silage biomass which produced higher amounts of biogas with an increased rate of production. Again, this demonstrates the beneficial effects of adding 11CH4 to forage prior to ensiling. However when untreated control silage is given to fermentations, synergistic improvements in gas production can be obtained by using a combination of X11M58 and 11CH4 added to the slurry.

1. A microbial inoculant comprising a combination of bacterial cultures and a suitable carrier, said combination comprising a first bacterial culture comprising Lactobacillus buchneri, or a derivative thereof, and a second bacterial culture comprising Bacillus licheniformis BL842, deposited as NRRL B-50516, Bacillus licheniformis BL21, deposited as NRRL B-50134, and Bacillus subtilis BS27, deposited as NRRL B-50105, or derivatives thereof.

2. The microbial inoculant of claim 1, wherein inoculation of forage with said combination of bacterial cultures prior to ensiling and biogas production results in a synergistic increase in production of biogas.

3. The microbial inoculant of claim 1, wherein inoculation of forage with said combination of bacterial cultures prior to ensiling results in a synergistic decrease in dry matter loss.

4. The microbial inoculant of claim 1, wherein said combination of bacterial cultures comprises equal parts of said first bacterial culture and said second bacterial culture.

5. The microbial inoculant of claim 1, wherein said second bacterial culture comprises:
about 70% Bacillus licheniformis BL842, deposited as NRRL B-50516, or a derivative thereof,
about 20% Bacillus licheniformis BL21, deposited as NRRL B-50134, or a derivative thereof, and
about 10% Bacillus subtilis BS27, deposited as NRRL B-50105, or a derivative thereof.

6. The microbial inoculant of claim 1, wherein said Lactobacillus buchneri is Lactobacillus buchneri deposited as Patent Deposit No. PTA-6138, or a derivative thereof.

7. The microbial inoculant of claim 1, wherein said carrier is a liquid or solid.

8. The microbial inoculant of claim 7, wherein said carrier comprises calcium carbonate, starch, or cellulose.

9. Forage comprising the microbial inoculant of claim 1.

10. The forage of claim 9 comprising from about 10¹ to about 10¹⁰ viable organisms of said first bacterial culture per gram of said forage and comprises from about 10¹ to about 10¹⁰ viable organisms of said second bacterial culture per gram of said forage.

11. The forage of claim 9 comprising from about 10³ to about 10⁶ viable organisms of said first bacterial culture per gram of said forage and comprises from about 10³ to about 10⁶ viable organisms of said second bacterial culture per gram of said forage.

12. The forage of claim 9, wherein said forage is grass, clover, maize, maize stover, alfalfa, rye, barley, oats, wheat, triticale, soybean, beans, sorghum, sun flower, radish, artichoke, peas, sugar beets, or any combination thereof.

13. (canceled)

14. The microbial inoculant of claim 1, wherein addition of said combination of bacterial cultures to a biomass composition comprising silage and sludge results in a synergistic increase in the production of biogas.

15. The microbial inoculant of claim 14, wherein said silage is produced from uninoculated forage.

16. The microbial inoculant of claim 14, wherein said combination of bacterial cultures comprises equal parts of said first bacterial culture and said second bacterial culture.

17. The microbial inoculant of claim 14, wherein said second bacterial culture comprises:
about 70% Bacillus licheniformis BL842, deposited as NRRL B-50516, or a derivative thereof,
about 20% Bacillus licheniformis BL21, deposited as NRRL B-50134, or a derivative thereof, and
about 10% Bacillus subtilis BS27, deposited as NRRL B-50105, or a derivative thereof.

18. The microbial inoculant of claim 14, wherein said Lactobacillus buchneri is Lactobacillus buchneri deposited as Patent Deposit No. PTA-6138, or a derivative thereof.

19. Sludge comprising the microbial inoculant of claim 14.

20. A biomass composition comprising sludge and silage, and further comprising the microbial inoculant of claim 14.

21. The biomass composition of claim 20, wherein said biomass composition comprises about 95% to about 99% of said sludge.

22. The biomass composition of claim 20 comprising from about 10⁴ to about 10⁷ viable organisms of said first bacterial culture per gram of said biomass composition and comprises from about 10⁴ to about 10⁷ viable organisms of said second bacterial culture per gram of said biomass composition.

23. A method of biogas production from silage comprising:
(a) adding an effective amount of a microbial inoculant to forage, wherein said microbial inoculant comprises a combination of bacterial cultures and a suitable carrier, said combination comprising a first bacterial culture comprising Lactobacillus buchneri, or a derivative thereof, and a second bacterial culture comprising Bacillus licheniformis BL842, deposited as NRRL B-50516, Bacillus licheniformis BL21, deposited as NRRL B-50134, and Bacillus subtilis BS27, deposited as NRRL B-50105, or derivatives thereof;
(b) ensiling said forage inoculated with said combination of bacterial cultures to produce silage; and
(c) adding biomass comprising said silage to a biogas generator, wherein biogas is produced in said biogas generator.

24. The method of claim 23, wherein the effective amount of said microbial innoculant results in a synergistic production of biogas.

25-37. (canceled)

38. A method of biogas production from a biomass composition comprising:
(a) combining silage and sludge to form a biomass composition;
(b) adding an effective amount of a microbial inoculant to said biomass composition, wherein said microbial inoculant comprises a combination of bacterial cultures and a suitable carrier, said combination comprising a first bacterial culture comprising Lactobacillus buchneri, or a derivative thereof, and a second bacterial culture comprising Bacillus licheniformis BL842, deposited as NRRL B-50516, Bacillus licheniformis BL21, deposited as NRRL B-50134, and Bacillus subtilis BS27, deposited as NRRL B-50105, or derivatives thereof;
(c) adding said biomass composition comprising said microbial inoculant to a biogas generator,
wherein biogas is produced in said biogas generator.

39-48. (canceled)

49. Biogas produced by the method of claim 23.

50. A method of reducing dry matter loss during ensiling comprising:
(a) adding an effective amount of a microbial inoculant to forage, wherein said microbial inoculant comprises a combination of bacterial cultures and a suitable carrier, said combination comprising a first bacterial culture comprising Lactobacillus buchneri, or a derivative thereof, and a second bacterial culture comprising Bacillus licheniformis BL842, deposited as NRRL B-50516, Bacillus licheniformis BL21, deposited as NRRL B-50134, and Bacillus subtilis BS27, deposited as NRRL B-50105, or derivatives thereof; and,
(b) ensiling said forage inoculated with said combination of bacterial cultures to produce silage,
wherein dry matter loss during ensiling of said inoculated forage is reduced when compared to dry matter loss from uninoculated forage.

51-58. (canceled)

59. Silage produced by the method of claim 50.

60. A biomass composition comprising the silage of claim 59, and further comprising sludge.

61-62. (canceled)
